(19) United States
(12) Patent Application Publication
(10) Pub. No.: US 2010/0268124 A1
(43) Pub. Date: Oct. 21, 2010

(54) METHOD AND APPARATUS FOR PROVIDING CONTRACTION INFORMATION DURING LABOUR

(76) Inventors: Emily Hamilton, Verdun (CA); Briglde Mattar, Town-of-Mount-Royal (CA)

Correspondence Address: LADAS & PARRY LLP, 26 WEST 61ST STREET, NEW YORK, NY, 10023, US

(21) Appl. No.: 12/747,022

(22) PCT Filed: Dec. 5, 2008
(86) PCT No.: PCT/CA08/02134

§ 371 Date: Jun. 9, 2010

Related U.S. Application Data
(60) Provisional application No. 61/012,619, filed on Dec. 10, 2007.

(51) Int. Cl. A61B 5/103; G06T 11/20

(52) U.S. Cl. 600/588; 345/440

(57) ABSTRACT

A method and apparatus for implementing a user interface for displaying uterine contraction information is provided. The contraction signal is processed to derive a sequence of contraction persistence indices. Each contraction persistence index in the sequence is associated to a portion of the contraction signal and conveys whether there is an excess in the rate of contraction in the associated portion of the contraction signal. If there is an excess in the rate of contraction in the associated portion of the contraction signal, the contraction persistence index conveys whether this excess is part of a sustained pattern of excess in the rate of contractions or whether the excess is likely to be transient. The sequence of contraction persistence indices is displayed to a user. Optionally, the graphical user interface is adapted for selectively causing an alarm event based at least in part on the sequence of contractility persistence indices. The method and apparatus may be implemented on any suitable computing platform such as for example desktop computers, laptop computers and personal digital assistants (PDAs).

### CROSS-REFERENCE TO RELATED APPLICATIONS

This application is related to:
U.S. patent application entitled “METHOD AND APPARATUS FOR PROVIDING CONTRACTION INFORMATION DURING LABOUR” filed on Mar. 9, 2007 by Emily Hamilton and which was assigned Ser. No. 11/716,496; and
U.S. application entitled “METHOD AND APPARATUS FOR DISPLAYING LABOUR RELATED INFORMATION ASSOCIATED TO AN OBSTETRICS PATIENT” filed on May 1, 2006 by Emily Hamilton and which was assigned Ser. No. 11/416,281.

In addition, for the purpose of the United States, this application claims the benefit of priority under 35 USC §120 based on U.S. provisional patent application Ser. No. 61/012,619 filed Dec. 10, 2007 by Emily Hamilton et al.

The contents of the above identified documents are incorporated herein by reference.

### FIELD OF THE INVENTION

The present invention relates generally to the field of obstetrics and, more specifically, to a method and apparatus for monitoring labor progression and for providing a user interface to display data conveying fetal and maternal information during labor.

### BACKGROUND

Uterine contractions are intermittent and co-ordinated tightenings of the uterine muscle. Uterine contractions provide the force that makes labour progress, by causing the baby to descend through the birth canal and making the cervix efface (shorten), and dilate (open). This force is related to the frequency, strength and duration of the contractions. Oxytocin is a natural hormone that causes uterine contractions. A synthetic version of oxytocin is often administered during labour to increase the frequency, duration and strength of uterine contractions or to induce labour. The medication is administered through a continuous intravenous infusion. There is no fixed dosage as in antibiotic therapy; rather the dose is adjusted frequently according to the patient's response to achieve the desired frequency and intensity of contractions.

When the uterine muscle contracts, the maternal blood vessels in it are constricted causing a temporary reduction in the blood flow and delivery of oxygen to the baby's placenta. Relaxation of the contraction restores the flow and oxygen delivery to the baby. In normal circumstances, babies tolerate contractions well. However, in other circumstances, such as when the placenta malfunctions or the contractions are excessively frequent with little or no relaxation time between them, the baby may not tolerate this reduction in oxygen delivery. If the situation remains uncorrected or worsens it may result in injury to the baby's brain and permanent disability.

At present, clinical staff estimates the frequency of contractions by feeling the mother's abdomen for a few minutes and noting the timing of a few contractions or by examining a paper tracing that shows a recording of contraction pressures/intensity over time. These assessments are performed periodically and the results recorded in the medical record.

A deficiency with the above-described methods for assessing contraction frequency is that they are prone to inaccuracy and incompleteness because they are visual estimates based on short selected segments of the tracing. Labour can last many hours. Fatigue, distraction and inexperience of the clinical staff can contribute to variable operator response. Frequent false alarms breed disregard. As a result, the caregiver may fail to make assessments at the prescribed time intervals and may fail to appreciate the degree and duration of the abnormality as well as the response of the baby. Thus, there can be a delay or failure to recognize overly frequent contractions, to adjust the medication correctly, resulting in an iatrogenic injury to the baby.

In the context of the above, there is a need to provide a method and device for monitoring contractions for an obstetrics patient that alleviates at least in part problems associated with the existing methods and devices.

### SUMMARY OF THE INVENTION

In accordance with a first broad aspect, the invention provides a method for displaying uterine contraction information. The method comprises receiving a contraction signal conveying information related to occurrences of uterine contractions over time and processing the contraction signal to derive a sequence of contraction persistence indices. Each contraction persistence index in the sequence is associated to a portion of the contraction signal and conveys whether there is an excess in the rate of contraction in the associated portion of the contraction signal. In cases where there is an excess in the rate of contraction in the associated portion of the contraction signal, the contraction persistence index conveys whether or not this excess is part of a sustained pattern of excess in the rate of contractions. The method also comprises causing the sequence of contraction persistence indices to be conveyed to a user on a display device.

In accordance with a specific example of implementation, each contraction persistence index in the sequence of contraction indices is derived at least in part based on:
i) a first contraction rate, the first contraction rate being associated to a first portion of the contraction signal; and
ii) a second contraction rate, the second contraction rate being associated to a second portion of the contraction signal, at least part of the second portion of the contraction signal preceding in time the first portion of the contraction signal.

In accordance with a specific example of implementation, the method also comprises causing an alarm event based at least in part on at least one contraction persistence index in the sequence of contraction persistence indices.

An advantage of the present invention is that it allows clinical staff to readily distinguish between short-term variations and/or excesses in the contraction rate and long term persistence of excess in the contraction rate. While short-term variations and/or excesses in the contraction rate could signal innocuous transient states, the clinical staff has a medical responsibility to review and possibly intervene such as by altering medication use or other suitable types of intervention in the case of long term persistence of excesses in the contraction rate is identified.

In accordance with another broad aspect, the invention provides an apparatus for displaying uterine contraction information in accordance with the above-described method.

In accordance with another broad aspect, the invention provides a computer readable storage medium storing a program element suitable for execution by a CPU, the program element implementing a graphical user interface module for displaying uterine contraction information. The graphical user interface module is adapted for displaying uterine contraction information in accordance with the above-described method.

In accordance with another broad aspect, the invention provides a labour monitoring system. The system includes a sensor for receiving information indicative of occurrences of uterine contractions over time. The system also includes an apparatus for implementing a user interface for displaying uterine contraction information in accordance with the above-described method. The system also includes a display unit in communication with the apparatus. The display unit is responsive to a signal releasing by the output of the apparatus to display the graphical user interface.

In accordance with yet another broad aspect, the invention provides a server system implementing a graphical user interface module for displaying uterine contraction information. The server system stores a program element for execution by a CPU. The program element includes a plurality of program element components. A first program element component is for receiving contraction signals conveying information related to occurrences of uterine contractions over time. A second program element component is for processing the contraction signal to derive a sequence of contraction persistence indices. Each contraction persistence index in the sequence is associated to a portion of the contraction signal and conveys whether there is an excess in the rate of contraction in the associated portion of the contraction signal. If there is an excess in the rate of contraction in the associated portion of the contraction signal, the contraction persistence index also indicates whether this excess is part of a sustained pattern of excess in the rate of contractions. A third program element component for generating and issuing a signal for displaying a graphical representation of the sequence of contraction persistence indices.

In accordance with yet another broad aspect, the invention provides a client-server system for implementing a graphical user interface module for displaying uterine contraction information. The client-server system comprises a client system and a server system operative to exchange messages over a data network. The server system stores a program element for execution by a CPU. The program element includes a plurality of program element components. A first program element component is for execution on the server system and is for receiving a contraction signal conveying information related to occurrences of uterine contractions over time. A second program element component is for execution on the server system and is for processing the contraction signal to derive a sequence of contraction persistence indices. Each contraction persistence index in the sequence is associated to a portion of the contraction signal and conveys whether there is an excess in the rate of contraction in the associated portion of the contraction signal. If there is an excess in the rate of contraction in the associated portion of the contraction signal, the contraction persistence index also indicates whether this excess is part of a sustained pattern of excess in the rate of contractions. The third program element component is to be executed on the server system and is for sending messages to the client system for causing the client system to display a graphical representation of the sequence of contraction persistence indices.

In a specific implementation, the client-server system includes a plurality of client systems operative to exchange messages with the server system over a data network. The data network may be of any suitable network configuration including Intranets and the Internet. The client systems may be embodied in any suitable computing device including, but not limited to, desktop computers, laptop computers and personal digital assistants (PDAs).

These and other aspects and features of the present invention will now become apparent to those of ordinary skill in the art upon review of the following description of specific embodiments of the invention in conjunction with the accompanying drawings.

### BRIEF DESCRIPTION OF THE DRAWINGS

In the accompanying drawings:

FIG. 1 shows a high-level functional block diagram of a labour monitoring system including an apparatus implementing a user interface for displaying uterine contraction information in accordance with a specific example of implementation of the present invention;

FIG. 2 is a functional block diagram of the apparatus implementing a user interface for displaying uterine contraction information shown in FIG. 1 in accordance with a specific example of implementation of the present invention;

FIG. 3 shows a specific example of implementation of a graphical user interface implemented by the apparatus shown in FIG. 1 for displaying uterine contraction information in accordance with a specific example of implementation of the invention;

FIGS. 4A and 4B show an alternative specific example of implementation of a graphical user interface implemented by the apparatus shown in FIG. 1 for displaying uterine contraction information in accordance with an alternative specific example of implementation of the invention;

FIG. 5A shows another alternative specific example of implementation of a variant of the graphical user interface implemented by the apparatus shown in FIG. 1 for displaying information related to maternal/fetal well-being and/or labour progression in accordance with another specific example of implementation of the invention;

FIG. 5B shows yet another alternative specific example of implementation of a variant of the graphical user interface implemented by the apparatus shown in FIG. 1 for displaying information related to maternal/fetal well-being and/or labour progression in accordance with another specific example of implementation of the invention;

FIGS. 6A and 6B are flow diagrams of a process for displaying uterine contraction information in accordance with a specific example of implementation of the present invention;

FIGS. 7A and 7B are graphical representations of contraction signals in accordance with non-limiting examples of implementation of the present invention;

FIG. 8 is a block diagram of an apparatus for providing uterine contraction information in accordance with a specific practical example of implementation of the present invention;

FIG. 9 is a high level conceptual block diagram of a program element for implementing a graphical user interface of the type shown in either one of FIGS. 3, 4A, 4B, 5A and 5B or the process depicted in FIGS. 6A and 6B in accordance with a specific example of implementation of the present invention;

FIG. 10 shows a functional block diagram of a client-server system for providing uterine contraction information in accordance in accordance with an alternative example of implementation of the present invention.

Other aspects and features of the present invention will become apparent to those ordinarily skilled in the art upon review of the following description of specific embodiments of the invention in conjunction with the accompanying figures.

### DETAILED DESCRIPTION

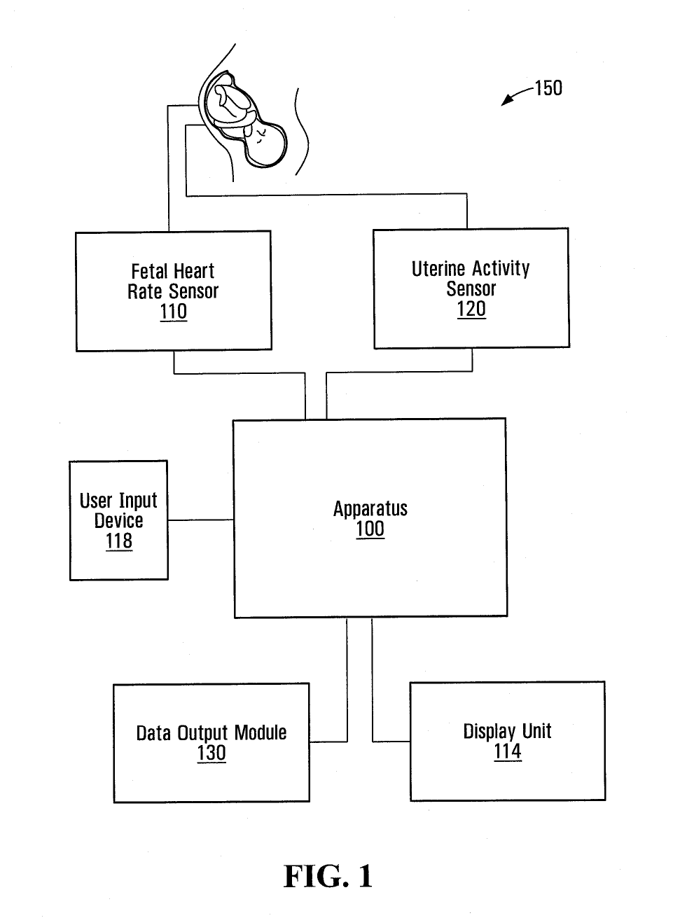

With reference to FIG. 1, there is shown a block diagram of a labour monitoring system 150 comprising a uterine activity sensor 120, a user input device 118, an apparatus 100 implementing a user interface for displaying uterine contraction information and a display unit 114.

In accordance with a specific implementation, the sensor 120 for monitoring uterine activity samples the contraction pattern at a certain pre-determined frequency to generate a signal indicative of uterine activity. The resulting signal, herein referred to as a contraction signal, conveys information related to the occurrence of uterine contractions over time. More specifically, the contraction signal conveys information on the occurrence of contraction events. Broadly stated, a contraction event refers to a continuous time period during which the uterine muscle of an obstetrics patient is tightening. During labour, contraction events are interleaved with relaxation periods during which the uterine muscle ceases to contract or contracts to a lesser extent. The contraction signal may be a continuous signal conveying contraction intensity information or may be comprised of unitary signal events where a signal event is generated when a contraction event is detected. Typically, when the contraction signal is comprised of unitary signal events, a signal event is generated when the onset of a contraction event is detected. Such sensors may be based on pressure or electrical signals from the abdominal wall, or within the uterine cavity. Sensors for monitoring uterine activity are well known in the art to which this invention pertains and any suitable sensor may be used without detracting from the spirit of the invention and as such will not be described further here.

Alternatively, certain embodiments of the labour monitoring system 150 may omit the sensor 120 and instead make use of a user-controlled input for generating the contraction signal. The user-controlled input allows a user to provide over time information signalling the onset of a contraction event such as to convey information associated to contraction activity over time. Such a user-controlled input may be in the form of a manually controlled actuator that can be activated by depressing a button when the obstetrics patient senses the onset of a contraction or in any other suitable configuration allowing a user to signal the onset of contraction events over time. Although the user controlling the actuator may be the expectant mother, it will most likely be a person other than the expectant mother, such as the expectant father or a nurse for example, since the expectant mother will most likely have other concerns during labour. In such an alternative embodiment, the contraction signal is comprised of unitary signal events. It will be readily appreciated that such a configuration may be somewhat inconvenient in practice since it would require that the user diligently enter contraction information. Consequently, although this alternative implementation has been presented for the purpose of completeness and as an alternative example of implementation, it will be readily appreciated that using a sensor 120 for monitoring uterine activity will be preferred in practical implementations of the invention.

The apparatus 100 is for implementing a graphical user interface module for displaying uterine contraction information. The contraction information may be displayed in various forms as will become apparent later on in the specification. Optionally, the graphical user interface module implemented by the apparatus 100 selectively causes an alarm event based at least in part on the uterine contraction information. The apparatus 100 also releases a signal for causing the display unit 114 to display the graphical user interface module. Optionally, the apparatus is further adapted for releasing signals to a data output module 130 for causing the latter to convey information related to maternal/fetal well-being and/or labour progression to a user of the labour monitoring system 150. Specific examples of implementation of the apparatus 100 and of the graphical user interface module will be described later on in the specification.

The user input device 118 is for receiving data from a user of the system. The user input device 118 may be used, for example, to enter information associated with the obstetrics patient and/or to manipulate the information displayed by the user interface implemented by the apparatus 100. Optionally still, the user input device 118 may be used to enter contraction medication information conveying information associated to administration of contraction inducing medication to the obstetrics patient. The contraction medication information may indicate whether contraction-inducing medication was administered and, optionally, the dosage of the contraction inducing medication that was administered. Since, typically, contraction-inducing medication is administered continuously over time and not as a one shot dose, the contraction medication information conveys the dosage of the contraction inducing medication administered over time. The user input device 118 may include any one or a combination of the following: keyboard, pointing device, touch sensitive surface, keypad or speech recognition unit. Certain embodiments of the labour monitoring system 150 may omit the user input device 118 without detracting from the spirit of the invention.

Optionally, as shown in FIG. 1, the labour monitoring system 150 may further include a fetal heart rate sensor 110. The fetal heart rate sensor 110 is for detecting a fetal heart rate of a fetus in-utero, also referred to as a fetus in the womb. The fetal heart rate sensor 110 samples the fetal heart rate at a certain pre-determined frequency to generate the signal indicative of the fetal heart rate. Fetal heart rate sensors are well known in the art to which this invention pertains and any suitable sensor for detecting a fetal heart rate may be used without detracting from the spirit of the invention and as such will not be described further here.

Optionally still, the labour monitoring system 150 may include other sensors (not shown) for measuring labour progress and the fetus' tolerance to labour. Such sensors may include for example:
a sensor for measuring the maternal oxygen saturation
a sensor for measuring the fetal oxygen saturation
a sensor for measuring maternal blood pressure
a sensor for measuring and analysing the fetal electrocardiogram

Such sensors are not critical to the invention and therefore will not be described further here.

The display unit 114 is in communication with the apparatus 100 and receives a signal causing the display unit 114 to display a graphical user interface module implemented by apparatus 100. The display unit 114 may be in the form of a display screen, a printer or any other suitable device for conveying to the physician or other health care professional uterine contraction information associated to an obstetrics patient. In embodiments where the display unit 114 is in the form of a display screen, it may be part of any suitable type of apparatus including, without being limited to, a desktop/laptop computing apparatus, a personal digital assistant (PDA), a telephone equipped to video display capability, a TV monitor or any other suitable device equipped with a display screen for visually conveying information to a user.

Optionally, the labour monitoring system 150 may further include a data output module 130. The data output module 130 is in communication with the apparatus 100 and is suitable for receiving signals generated by the apparatus 100. In a first specific example of implementation, the data output module 130 includes an audio module for releasing audio signals on the basis of signals received from the apparatus 100. In a second specific example of implementation, the data output module 130 includes a data communication entity suitable for transmitting messages to remote devices causing the latter to convey to a user of the labour monitoring system 150 information related to maternal/fetal well-being and/or labour progression. Examples of remote devices include, without being limited to, PDAs, telephones, pagers and computing terminals.

A specific practical implementation of the labour monitoring system 150 may implement the graphical user interface module for displaying uterine contraction information as a stand-alone component or alternatively as part of a more complete labour monitoring system including a plurality of modules for monitoring various aspects of maternal/fetal well-being and/or labour progression. An example of such a labour monitoring system is described in co-pending U.S. patent application entitled “METHOD AND APPARATUS FOR DISPLAYING LABOUR RELATED INFORMATION ASSOCIATED TO AN OBSTETRICS PATIENT” filed on May 1, 2006 by Emily Hamilton and which was assigned Ser. No. 11/416,281, and was published on Nov. 1, 2007 under U.S. publication No. 20070255588A1. The contents of the above application are incorporated herein by reference.

### Apparatus 100

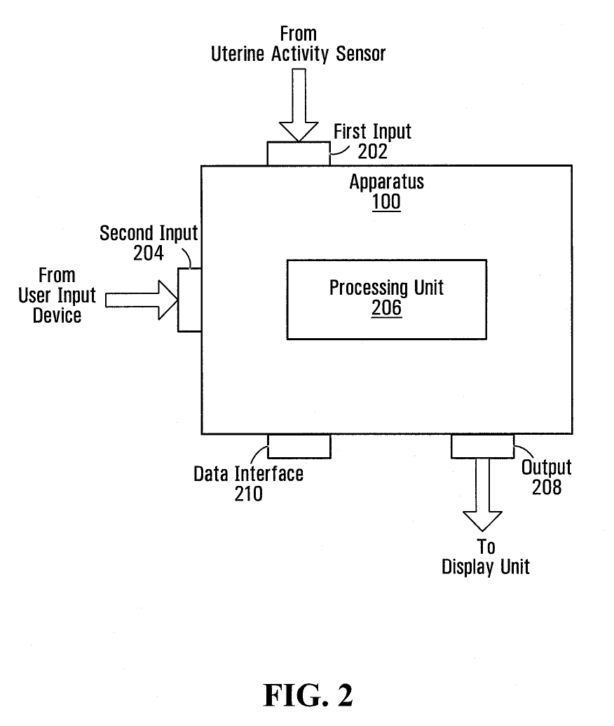

A specific example of implementation of apparatus 100 will now be described with reference to FIG. 2. The apparatus 100 includes an input 202 (labelled as first input in the figure), a processing unit 206 and an output 208. The first input 202 is for receiving a contraction signal originating from the uterine activity sensor 120 (shown in FIG. 1) and conveying information related to occurrences of uterine contractions over time. The processing unit 206 is in communication with the first input 202 and implements a graphical user interface for displaying uterine contraction information. The output 208 is for releasing a signal for causing the display unit 114 (shown in FIG. 1) to display the graphical user interface implemented by processing unit 206. Optionally, as shown in FIG. 2, the apparatus further includes a second input 216 for receiving data from a user through input device 118 (shown in FIG. 1). Optionally still, the apparatus further includes a data interface 210 for exchanging signals with a data output module 130 (shown in FIG. 1) for causing the latter to convey information related to maternal/fetal well-being and/or labour progression to a user of the labour monitoring system 150 (shown in FIG. 1).

Optionally, the apparatus further includes an additional input (not shown in the figures) for receiving fetal heart rate information. The fetal heart rate information may including a fetal heart rate signal as generated by fetal heart rate sensor (110) or, alternatively, may include information conveying a level of risk associated with the fetus, the level of risk being derived on the basis of a fetal heart rate signal. Where the fetal heart rate information includes a fetal heart rate signal, the apparatus 100 is adapted for processing the signal to determine a level of risk associated with the fetal heart rate signal. Any suitable method for assessing a level of risk on the basis of a fetal heart rate signal may be used. For example, the level of risk may be based on the frequency of the fetal heart rate, whether it is too high or too low for a certain period of time. Alternatively, the level of risk may be based on other known methods. A non-limiting example of a method for providing an indication of the level of risk is described in U.S. Pat. No. 7,113,819, entitled “Method and apparatus for monitoring the condition of a fetus”, issued on Sep. 26, 2006 to E. Hamilton et al. and assigned to LMS Medical Systems Ltd. The contents of this document are incorporated herein by reference. Other suitable methods for assessing a level of risk on the basis of a fetal heart rate signal may be used without detracting from the spirit of the invention.

The graphical user interface module implemented by apparatus 100 will now be described in greater detail.

The graphical user interface module receives the contraction signal originating from input 202 and conveying information related to uterine contractions over time. The graphical user interface module processes the contraction signal to derive a sequence of contraction persistence indices. Each contraction persistence index conveys whether there is an excess in the rate of contractions in an associated portion of the contraction signal and whether this excess is part of a sustained pattern of excess in the rate of contractions. The graphical user interface module then conveys the sequence of contraction persistence indices to a user. Optionally, the graphical user interface module displays, concurrently with the contractility persistence index, information conveying rates of uterine contractions over time and/or information conveying a threshold rate of uterine contractions.

Generally stated, the contractility persistence index is an index intended to capture one or more characteristics of the contraction signal, in particular excesses in the rate of contractions and the duration of these excesses. In this manner, the contractility persistence index combines both contraction frequency and duration information into a same information element in order to provide the clinical staff with an indication of whether an excess in the rate of contraction is sustained or is merely transient.

In a specific example, the contractility persistence index assigned to time “n” is a function of the contraction rate at time “n” and of the contraction rate at one or more times preceding time “n”. Mathematically, a specific example of the contractility persistence index can be expressed as follow:

$$Contractility Persistence Index [ n ] = f ( contraction rate [ n ] , contraction rate [ n - {k}_{1} ] , … , contraction rate [ n - {k}_{m} ] ) \\ 0 < {k}_{1} {…k}_{m} \leq n \\ where {k}_{1} {…k}_{m} are integers \qquad ( 1 )$$

where Contractility Persistence Index [n] denotes the contractility persistence index at time “n”; contraction rate[n] denotes the rate of contraction at time “n”; contraction rate [n−kᵢ] and contraction rate [n−kₘ] denotes the rate of contraction at one or more times preceding time “n” and f( ) denotes a function. The above equation denotes the computation of the contractility persistence index as a value calculated at discrete instants in time over time. It will be readily appreciated that, in certain implementations, the contractility persistence index may be computed in a continuous fashion over time without detracting from the spirit of the invention.

The nature of function f( ) used for determining the contractility persistence index at a certain time may vary from one implementation to the other. The function f( ) may be a mathematical function or may simply denote a set of rules applied to determine the value of the contraction persistence index to assign at a given time.

For the purpose of illustration, specific examples of contraction persistence indices will now be described.

As a first practical example of implementation, the contractility persistence index at a time “n” is based on a weighted sum of the contraction rate at time “n” and of one or more contraction rate(s) at times preceding time “n”. Mathematically, a specific example of such a contractility persistence index can be expressed as follow:

$$\begin{matrix}Weighted Sum [ n ] = {w}_{0} \times contractionrate [ n ] + \\ {\sum }_{i = 1}^{m} {w}_{i} \times contractionrate [ n - {k}_{i} ]\end{matrix} \\ 0 < {k}_{i} \leq n \\ where {k}_{i} is an integer \\ 0 \leq {w}_{0} and {w}_{i} \leq 1 \\ and \\ Contractility Persistence Index [ n ] = G ( Weighted Sum ) \qquad ( 2 )$$

where Weighted Sum[n] denotes the weighted sum of the contractions at time “n”; contraction rate[n] denotes the rate of contraction at time “n”; contraction rate [n−kᵢ] for denotes the rate of contraction at one or more times preceding time “n”; w₀ and wᵢ are weight values assigned to the contraction rates measured at different times; the Contractility Persistence Index [n] denotes the contractility persistence index at time “n” and G( ) denotes a function. In a specific example, contraction rates at times further from time n are weighted less heavily than contraction rates at times closer to time n. In its simplest form, the G( ) is the identify function, in other words the weighted sum is itself the index. In another example, the function G( ) provides a mapping between different possible values of the weighted sum and a set of index levels. In another example, the function G( ) is an averaging function so that the contractility persistence index is a weighted average of the contraction rates over time.

Other embodiments of the contractility persistence index may take into account whether the contraction rate exceeds a threshold contraction rate. The threshold rate of uterine contractions defines boundaries of safe care and may be set in accordance best practices or in accordance with hospital/care-giver facility policy. Although most the present description will typically refer to a single threshold rate of uterine contraction, it will be readily apparent that embodiments including multiple thresholds of uterine contractions, each threshold being associated with a respective degree of risk to the obstetrics patient, may be used in alternative implementations of the present invention.

As a second practical example, the contractility persistence index includes four levels defined as follows:

Contractility persistence index [n]=
Level 1: If the contraction rate at time n is below the threshold contraction rate;
else
Level 2: The contraction rate at the current time is above the threshold contraction rate and has been so for less than a first predetermined time duration (e.g. 10 min);
else
Level 3: The contraction rate at time n is above the threshold contraction rate and has been so for longer that the first predetermined time duration (e.g. 10 min) but longer that the second predetermined time duration (e.g. 20 min);
else
Level 4: The contraction rate at time n is above the threshold contraction rate and has been so for longer than the second predetermined time duration (e.g. 20 min).

In the example above, the first and second predetermined time durations may be established on the basis of a hospital policy or, alternatively, on the basis of other clinical guidelines and may therefore vary from one implementation to the other. Although in the above example the contractility persistence index only includes four (4) levels, it will be readily apparent that specific implementations may include fewer or additional levels without detraction from the spirit of the invention. Other embodiments of the above contractility persistence index may make use of multiple threshold contraction rates and/or may take into account the degree or extent to which the contraction rate exceeds a threshold contraction rate.

Other embodiments of the contractility persistence index may take into account the degree or extent to which the contraction rate exceeds a threshold contraction rate. As a third specific example, such a contractility persistence index is presented below:

$$\begin{matrix}Weighted Sum [ n ] = {w}_{0} \times \{ contractionrate [ n ] - threshold \} + \\ {\sum }_{i = 1}^{m} {w}_{i} \times \{ contractionrate [ n - {k}_{i} ] - threshold \}\end{matrix} \qquad ( 3 )$$

As will be appreciated by the reader skilled in the art, the above described examples of contractility persistence indices are only examples and many possible variants and alternatives are possible.

In particular, although the above described examples of contractility persistence indices have only considered factors related to the contraction signal, it will be appreciated that variants of the contractility persistence index may consider other labour related progress in addition to the contraction signal. Example of other factors that may be considered in the contractility persistence index include, without being limited to, fetal heart rate (FHR) abnormalities and the level of uterotonic medication (such as oxytocin) administered to the obstetrics patient.

In a specific example of implementation, the contractility persistence index value is derived for each time segment of duration T and is displayed in graphical format. In a non-limiting example of implementation, the duration T of the time segment is 2 minutes.

The specific manner in which the information related to the contractility persistence index can be displayed to a user of the system 150 by the graphical user interface module may vary from one implementation to the other without detracting from the spirit of the invention.

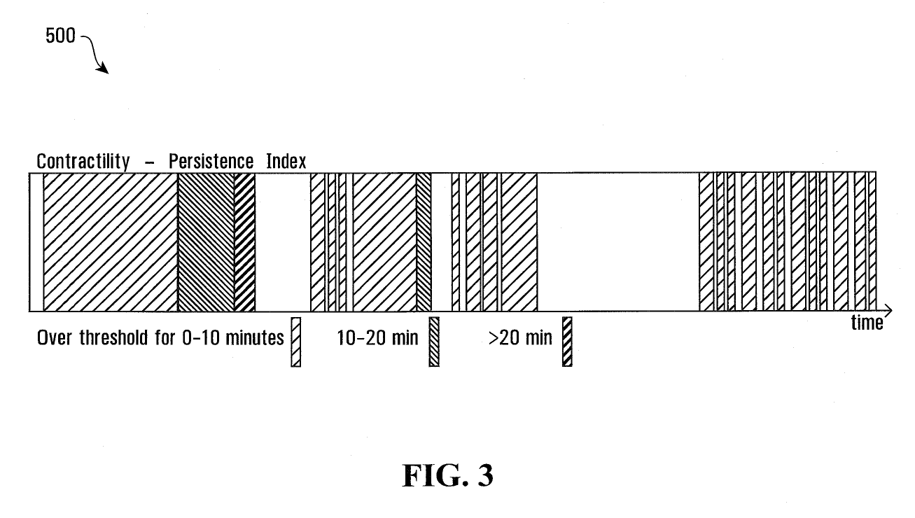

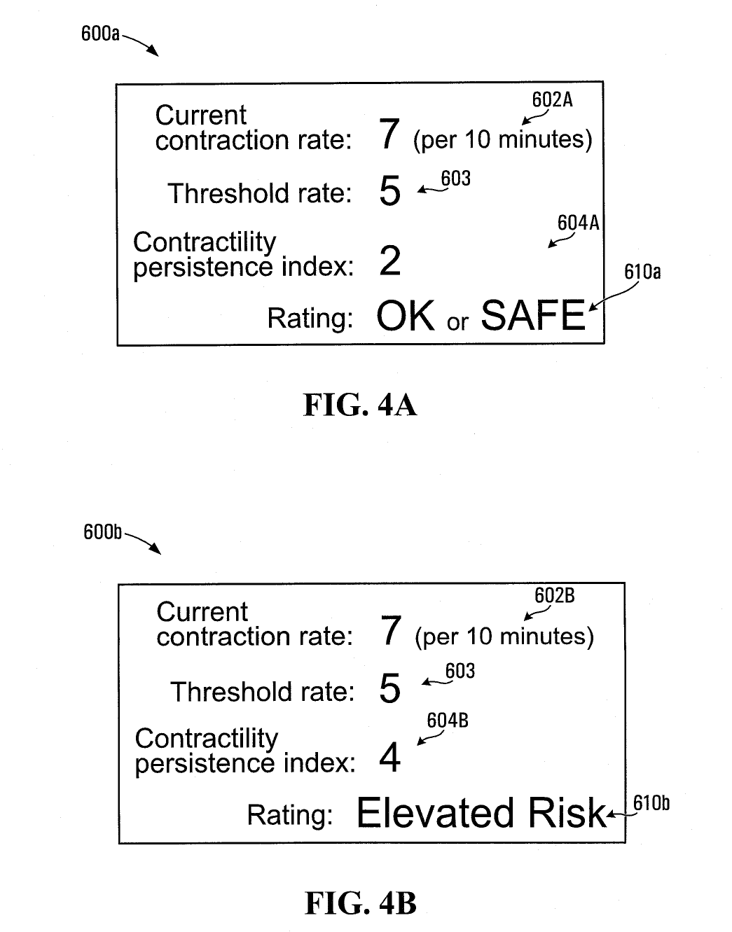

Specific non-limiting examples of implementation of a graphical user interface module are shown in FIGS. 3, 4A and 4B.

A first specific example of implementation of the graphical user interface module is shown in FIG. 3 of the drawings.

In this specific implementation, the value of the contractility persistence index is displayed as a function of time (time is shown in the horizontal axis). In the example shown in FIG. 3, bands of different colours are used to convey different values of the index over time. More specifically, a different color (or color shade) is associated to a respective value, or range of values, of the contractility persistence index. Referring back to the second practical example described previously, where the contractility persistence index includes four levels, namely Level 1, Level 2, Level 3 and Level 4, a specific example of a color scheme, and the one depicted in FIG. 3, is white for level 1 and colored for each of levels 2-4 where the color intensity increases for each increase in level. Other suitable manners for conveying different values of the index over time may be used instead of a colour scheme. Specific examples include, without being limited to a bar chart or line graph where a different height is associated to a respective value, or range of values, of the contractility persistence index.

Advantageously, the implementation depicted in FIG. 3 allows the clinical staff to readily observe the trend in contraction rate over time for a given obstetrics patient and to easily assess whether an excess in the contraction rate is sustained and transitory. This may allow the clinical staff to ascertain more easily whether the excess is minor, indicating perhaps a low level risk, or whether it is significant, requiring a quicker intervention.

A second specific example of implementation of the graphical user interface module is shown in FIGS. 4A and 4B of the drawings. In this example, the previously described second practical example of the contractility persistence index is being used, where the contractility persistence index includes four levels, namely Level 1, Level 2, Level 3 and Level 4. In the specific implementation shown in FIG. 4A, the information displayed includes a first alphanumeric element 602a conveying a current rate of uterine contractions associated to an obstetrics patient, a second alphanumeric element 603 conveying a threshold rate of uterine contractions and a third alphanumeric element 604a conveying the value of the contractility persistence index. In this specific implementation, the first alphanumeric element 602a reflects the current contraction rate derived on the basis of a contraction signal received at input 202 (shown in FIG. 2). In the non-limiting example shown in FIG. 4A, the first alphanumeric element 602a conveys the number of contraction events over the previous 10 minutes. The third alphanumeric element 604a reflects the current value of the contractility persistence index. The first and third alphanumeric element 602a 604a are continuously, or periodically, updated over time on the basis of the contraction signal receiving at input 202 (shown in FIG. 2). The threshold rate of uterine contractions conveyed by the second alphanumeric element 603 indicates the boundary between uterine contraction rates considered to be within safe boundaries and contraction rates considered as being associated to riskier situations. In the example depicted, the second alphanumeric element 603 conveys a contraction rate of 5 contractions/10 minutes, corresponding to a common definition of uterine hypercontractility. It will be readily appreciated that other suitable threshold rates of uterine contractions may be used without detracting from the spirit of the invention.

In FIG. 4B, the same example of implementation of the graphical user interface module as that shown in FIG. 4A is shown but the third alphanumeric element 604b indicates a different value for the contractility persistence index. More specifically, as illustrated in FIGS. 4A and 4B, the current contraction rate conveyed by the first alphanumeric elements 602a and 602b is 7 contractions per 10 minutes and the threshold rate is set to 5 contractions per 10 minutes. As such, in both FIGS. 4A and 4B, the current contraction rate exceeds the threshold rate. However, in FIG. 4A, the third alphanumeric element 604a indicates Level 2 as the value of contractility persistence index. This information indicates that the contraction rate at the current time is above the threshold contraction rate but has been so for less than a first predetermined time duration (e.g. less than 10 min). As such, for the time being, the clinical staff could interpret the information on the display 600a to mean that although the current contraction rate exceeds the threshold rate, this excess can be considered transitory for the time being. Conversely, in FIG. 4B, the third alphanumeric element 604b indicates Level 4 as the value of contractility persistence index. This information indicates that the contraction rate at the current time is above the threshold contraction rate and has been so for more than a second predetermined time duration (e.g. more than 20 min). As such, for the time being, the clinical staff would interpret the information on the display 600b to mean that the current contraction rate exceeds the threshold rate and this excess appears to be sustained and that intervention should be considered.

Advantageously, in situations where the current contraction rate exceeds the boundaries of safe care as conveyed by the second alphanumeric element 603, the third alphanumeric element 604a, which reflects the current value of the contractility persistence index, provides an indicating of whether this excess is transitory and therefore not critical or sustained and potentially requiring intervention.

Optionally, as depicted in the specific examples shown in FIGS. 4A and 4B, the graphical user interface module also displays an alphanumeric indicator 610a 610b in the form of a rating. In the example depicted in FIG. 4A, the third alphanumeric element 604a indicates Level 2 as the value of contractility persistence index. In this case the alphanumeric indicator 610a indicates the message “OK or SAFE” conveying that the excess in the current contraction rate is considered likely transitory for the time being. In FIG. 4B, the third alphanumeric element 604b indicates Level 4 as the value of the contractility persistence index. In this case the alphanumeric indicator 610b indicates the message “ELEVATED RISK” conveying that the excess in the current contraction rate is considered sustained raising the risk level for the obstetric patient. In alternative examples of implementation, the alphanumeric indicator 610b may be adapted for displaying graded risk levels such as for example “mildly elevated”, “moderately elevated” and “critically elevated” for example.

### Alarm Events

The graphical user interface module is adapted for selectively causing an alarm event based at least in part on the contractility persistence index. In a specific example of implementation, the alarm event is for alerting the clinical staff making use of the system of an occurrence of a potentially problematic situation during labour associated to the occurrence of contractions. The alarm event may be triggered in a number of situations and may be based on the contractility persistence index and optionally on the basis of either one or both of contraction medication information and fetal heart rate information. Examples of the manners in which an alarm event may be selectively caused will be described later on in the specification.

An alarm event, in accordance with a specific example of implementation of the invention, may include one or more components for communicating information to a user of the graphical user interface module.

In a first specific implementation, the alarm event includes displaying a visual indicator to convey to a user of the graphical user interface module an occurrence of a potentially problematic situation during labour. The visual indicator may be displayed as part of the graphical user interface module or in a separate display at a remote location. Any suitable type of visual indicator may be used. Examples of visual indicators that may be used include, without being limited to:
Variations in color. For example, a color scheme may be established whereby certain colors are associated with varying levels of risk. Portions of the graphical user interface may turn a certain color associated with a high level of risk when, for example, the contractility persistence index is above a certain limit. In a non-limiting example, the entire display window or a portion of the window may be displayed in a certain color associated with a high level of risk based at least in part one the value of the contractility persistence index. A non-limiting example of a color scheme is green=normal; yellow=intermediate risk level; red=high level of risk. It is to be appreciated that any suitable color scheme may be used.
Variations in the display intensity of the viewing window. For example, flashing or blinking of the viewing window may be used as a visual indicator to draw the attention of the user;
Variations in the size or position of the viewing window. For example, the viewing window may be made to appear more prominently on the display unit or at a location that is more likely to draw the attention of the clinical staff;
Displaying a message prompting/alerting the clinical staff. For example, in FIG. 4B, an alphanumeric message 610 is displayed as “ELEVATED RISK” to convey that the excess in the current contraction rate is considered sustained raising the risk level for the obstetric.

In a second specific implementation, the alarm event includes causing an audio signal to be issued, alone or in combination with a visual indicator, to draw attention of a user of the graphical user interface module. In this second specific implementation, the processing unit 206 (shown in FIG. 2) releases a signal at the data interface 210 for causing an audio unit (not shown in the figures) to issue an audio signal. The audio unit may be connected directly to the data interface 210 through either a wire-line link or a wireless link. Alternatively, the audio unit may be in communication with the data interface 210 over a network. Alternatively still, the audio unit may be an integral part of apparatus 100.

In a third specific implementation, the alarm event includes causing a message signal to be transmitted to a remote device. The remote device may be, for example, a PDA, telephone, pager or a remote computing terminal. Other suitable types of remote devices may also be envisaged in other specific implementations of the present invention. In this third specific implementation, the processing unit 206 (shown in FIG. 2) releases a signal at the data interface 210 for causing a message signal to be transmitted to the remote device. The remote device may be connected directly to the data interface 210 though either a wire-line link or a wireless link. Alternative, the remote device may be in communication with the data interface 210 over a network.

In a first practical example of interaction, the remote device is a PDA assigned to a doctor responsible for overseeing deliveries in a hospital. At least in part based on the value of the contractility persistence index, the graphical user interface module selectively sends a message through the data interface 210 and over a network to the PDA of the doctor to alert that doctor. The message may include any suitable useful information including, but not limited to, the name of the obstetrics patient, the location of the patient, the contraction rate, the value of the contractility persistence index, contraction medication information, fetal heart rate information, labour progression information (duration of labour, time since admission to hospital) and medical history. Optionally, the message may also enable the PDA of the doctor to display all or part of the user interface module described in the present application. For example, the message may enable the PDA of the doctor to display a user interface of the type depicted in FIGS. 3, 4A and 4B. Alternatively, the message may only indicate that a certain patient requires closer monitoring of her contraction rate. The specific format of the message is not critical to the invention and as such will not be discussed further here.

In second practical example of interaction, the remote device is a remote computing terminal located at a centralised nursing station in a hospital birthing centre. At least in part on the value of the contractility persistence index, the graphical user interface module selectively causes a message to be sent to the remote computing terminal. Advantageously, by allowing a message to be transmitted to a remote device, the clinical staff need not be located near the patient or in proximity to the patient to be alerted to potentially problematic situations. In addition, the clinical staff need not be expressly monitoring the progression of the contraction rate to be alerted to an unsafe condition.

### The Process

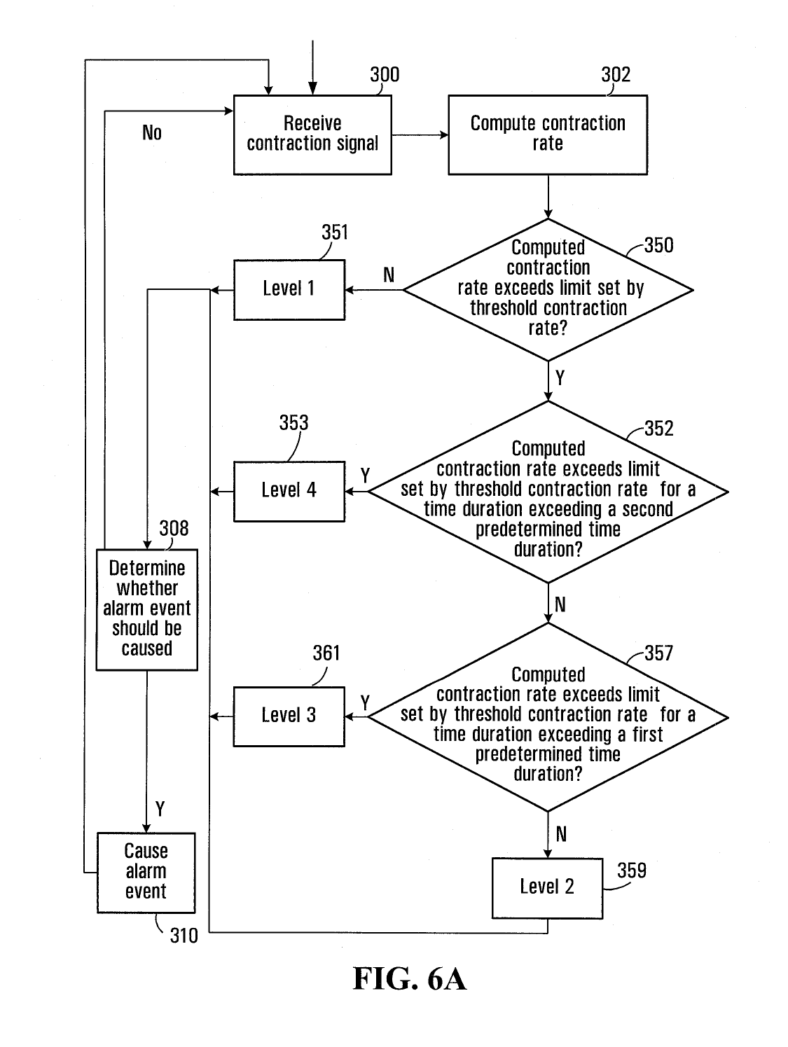

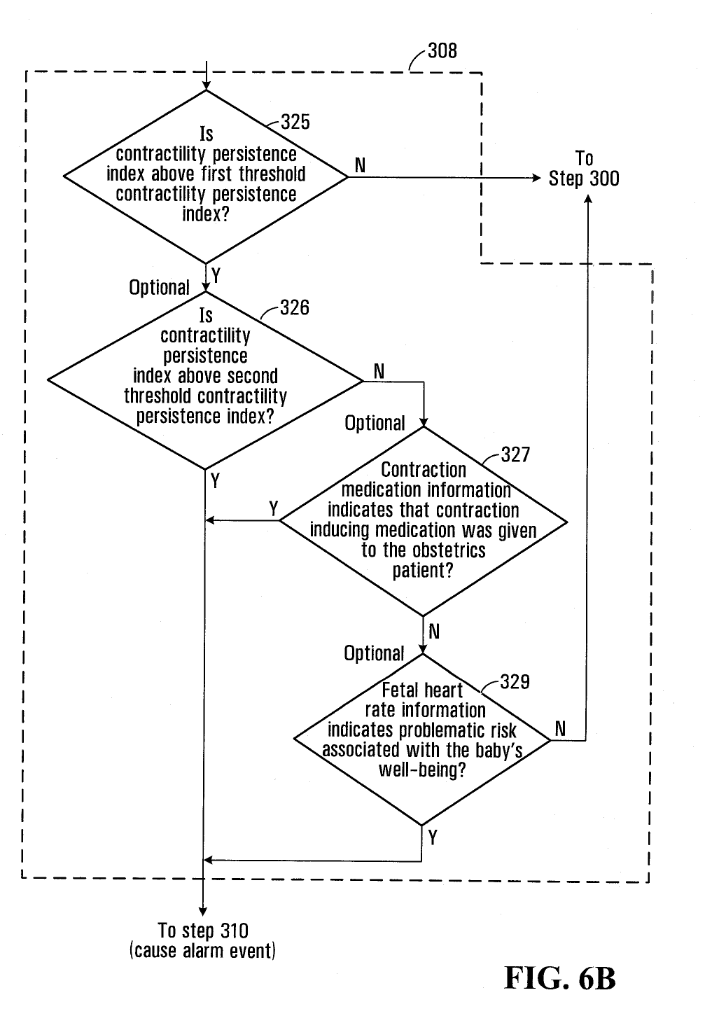

An exemplary embodiment of the process implemented by the graphical user interface will now be described with reference to FIGS. 6A and 6B.

For the purpose of this example, the contractility persistence index includes four levels defined as follows:
Level 1: If the contraction rate at time n is below the threshold contraction rate;
else
Level 2: The contraction rate at the current time is above the threshold contraction rate and has been so for less than a first predetermined time duration (e.g. 10 min);
else
Level 3: The contraction rate at time n is above the threshold contraction rate and has been so for longer that the first predetermined time duration (e.g. 10 min) but longer that the second predetermined time duration (e.g. 20 min);
else
Level 4: The contraction rate at time n is above the threshold contraction rate and has been so for longer than the second predetermined time duration (e.g. 20 min).

With reference to FIG. 6A, at step 300, the contraction signal is received by the graphical user interface module.

At step 302, the graphical user interface module computes a contraction rate on the basis of the contraction signal received at step 300.

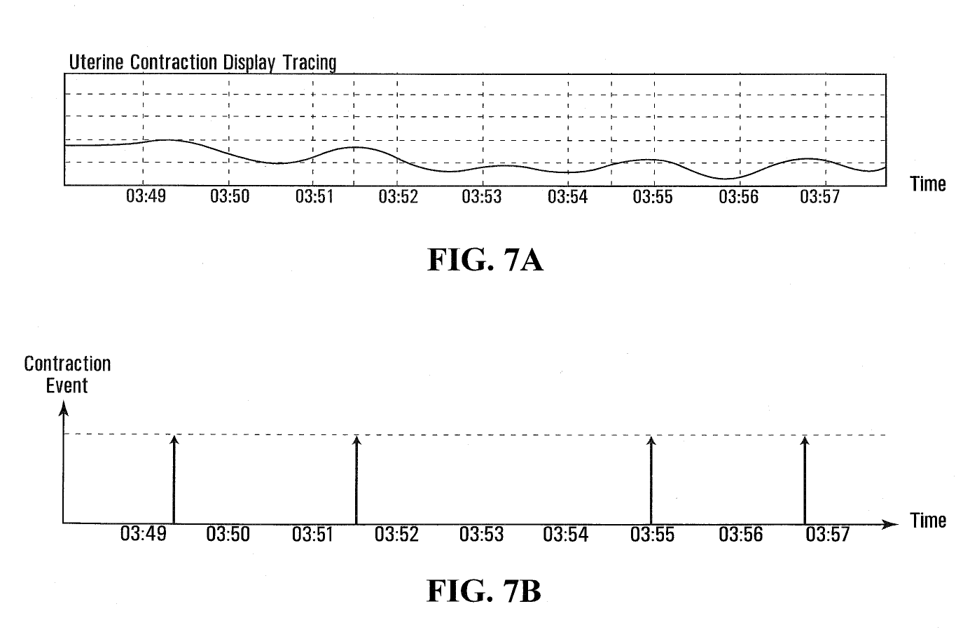

The specific manner in which the contraction rate is computed will depend on the format of the contraction signal. In a first specific example, the contraction signal is a continuous signal conveying the intensity of the uterine contractions over time. A non-limiting graphical representation of such a continuous signal is depicted in FIG. 7A for the purpose of illustration. In such an implementation, the graphical user interface module is operative for processing the contraction signal to detect the occurrence of contraction events in the contraction signal. Any suitable pattern recognition technique may be used for identifying the occurrence of contraction events. Such techniques are well known in the art of signal processing and as such will not be described further here. Once the occurrence of contraction events has been detected, the contraction rate can be computed.

In a second specific example, the contraction signal received at input 202 is comprised of unitary signal events where a signal event is generated when a contraction event is detected. A non-limiting graphical representation of such a continuous signal is depicted in FIG. 7B for the purpose of illustration. In such an implementation, pattern recognition techniques are not required since the presence of contraction events is already conveyed by the contraction signal.

In a specific implementation, the graphical user interface module computes a rate of contraction events in the contraction signal for a certain time segment. The rate of contraction events in the contraction signal may be computed in a number of suitable manners.

In a specific example, a current contraction rate is equal to the number of contraction events detected in the contraction signal over the last time duration T. The duration T may be any suitable time duration. In a non-limiting example, the duration T is 10-15 minutes and the current contraction rate is the number of contraction events in the contraction signal that occurred over the previous 10-15 minutes. Most clinical guidelines describe the desirable contraction frequency based on an observation period of 10-15 minutes. It will be readily apparent to the person skilled in the art that the time duration T may have a duration different than 10-15 minutes. Moreover, the time duration T may be a configurable parameter of the graphical user interface module implemented by processing unit 206 without detracting from the spirit of the invention. Typically, the duration T will be selected to be a time duration sufficiently long so that a few contraction events are likely to occur during active labour but sufficiently short so that the contraction rate for a given time duration T is representative of the progression of the contraction rate during active labour. It will be readily apparent to the person skilled in the art that a very lengthy time duration, let us say 3 hours, does not provide useful information as to whether the contraction rate is within reasonable boundaries. Similarly, a very short time duration, let us say 2 minutes, also does not provide any useful information as to whether the contraction rate is within reasonable boundaries.

It will be readily apparent to the person skilled in the art, in light of the present description, that other well-known techniques for computing a contraction rate on the basis of a contraction signal may be used without detracting from the spirit of the invention.

In a specific example of implementation, the contraction rate is computed every 2 minutes for the previous 10 minutes time segment. In other words, the contraction rate is expressed as the number of contraction for a 10-minute time interval and is computed every 2 minutes for the preceding 10-minute time interval.

At step 350 the system performs a comparison to determine whether the computed contraction rate exceeds the limit set by a threshold contraction rate. The threshold contraction rate is a configurable parameter in the system and will typically be set by hospital policies or best medical practice rules. If step 350 is answered in the negative and the computed contraction rate does not exceed the limit set by threshold contraction rate, the system proceeds to step 351 where the contractility persistence index for that time interval is set to Level 1. The system then displays that information graphically and proceeds to optional step 308. If optional step 308 is omitted from the implementation, the system returns to step 300 to process the next time interval.

If step 350 is answered in the affirmative and the computed contraction rate exceeds the limit set by threshold contraction rate, the system proceeds to step 352 where an additional condition is tested.

At step 352 the graphical user interface module determines whether the computed contraction rate has exceeded the limit set by the threshold contraction rate for a time duration exceeding a second predetermined time duration. This step 352 allows testing whether the excess of the contraction rate is merely transient of whether it is sustained. The predetermined time duration may be established on the basis of a hospital policy or, alternatively, on the basis of other clinical guidelines. In a non-limiting implementation, the time duration used in at step 352 is 20 minutes.

If step 352 is answered in the affirmative and the computed contraction rate exceeds the limit set by threshold contraction rate for a duration of time exceeding the predetermined time duration of 20 minutes, the system proceeds to step 353 where the contractility persistence index for that time interval is set to Level 4. The system then displays that information graphically and proceeds to optional step 308. If optional step 308 is omitted from the implementation, the system returns to step 300 to process the next time interval.

If step 352 is answered in the negative and the computed contraction rate has not exceeded the limit set by threshold contraction rate for a duration of time exceeding 20 minutes, the system proceeds to step 357 where an additional condition is tested.

At step 357 the graphical user interface module determines whether the computed contraction rate has exceeded the limit set by the threshold contraction rate for a time duration exceeding a first predetermined time duration. In a non-limiting implementation, the first predetermined time duration used at step 357 is 10 minutes.

If step 357 is answered in the affirmative and the computed contraction rate exceeds the limit set by the threshold contraction rate for a duration of time exceeding the predetermined time duration of 10 minutes, the system proceeds to step 361 where the contractility persistence index for that time interval is set to Level 3. The system then displays that information graphically and proceeds to optional step 308. If optional step 308 is omitted from the implementation, the system returns to step 300 to process the next time interval.

If step 357 is answered in the negative and the computed contraction rate has not exceeded the limit set by threshold contraction rate for a duration of time exceeding 10 minutes, the system proceeds to step 359 where the contractility persistence index for that time interval is set to Level 2. The system then displays that information graphically and proceeds to optional step 308. If optional step 308 is omitted from the implementation, the system returns to step 300 to process the next time interval.

Specific non-limiting examples of formats for the display of the information were described with reference to FIGS. 3, 4A and 4B of the drawings.

Step 308 is an optional step and may be omitted from certain specific implementations. At step 308, the graphical user interface module determines, at least in part on the basis of the computed contractility persistence index, whether an alarm event should be caused.

As will become apparent to the person skilled in the art in light of the present specification, different conditions may bring the graphical user interface module to cause an alarm event.

In a first specific example of implementation, an alarm event is triggered depending on the specific circumstances conveyed by the contractility persistence index alone.

In a second specific example of implementation, an alarm event is triggered depending on the specific circumstances conveyed by the contractility persistence index in combination with other factors. Such other factors may include, without being limited to, contraction medication information and fetal heart rate information.

In either one of the above described specific examples of implementation, the conditions for causing an alarm event may be determined on the basis of a hospital policy or in accordance with best recognised practices in health care.

In a specific example of implementation, step 308 shown in FIG. 6A includes multiple sub-steps for determining whether an alarm event should be caused. FIG. 6B shows a non-limiting example of implementation of process step 308.

As depicted, at step 325 the graphical user interface module determines whether the contractility persistence index exceeds the limit set by a first threshold contractility persistence index. If step 325 is answered in the negative and the computed contractility persistence index does not exceed the limit set by the first threshold contractility persistence index, step 308 determines that no alarm should be caused and the graphical user interface proceeds to step 300.

If step 325 is answered in the affirmative and the computed contractility persistence index exceeds the limit set by the first threshold contractility persistence index, the graphical user interface proceeds to step 326 where an additional condition is tested. In a specific example, the first threshold contractility persistence index is set to Level 3. As such, a contractility persistence index having a value of Level 3 or higher will be further considered to determine whether an alarm should be caused.

At step 326 the graphical user interface module determines whether the computed contractility persistence index has exceeded the limit set by a second threshold contractility persistence index. This step 326 allows testing the severity of the persistence of an elevated contraction rate. In a specific example, the second threshold contractility persistence index is set to Level 4.

If step 326 is answered in the affirmative and the computed contractility persistence index exceeds the limit set by the second threshold contractility persistence index indicating that the excess of the contraction rate is sustained and severe, step 308 determines that an alarm event should be caused and the graphical user interface proceeds to step 310 where an alarm event is caused to occur. As such, in the present specific example of implementation, a contractility persistence index having a value of Level 4 will be considered severe enough to cause an alarm on its own without considering other factors.

If step 326 is answered in the negative and the computed contractility persistence index has not exceeded the limit set by the second threshold contractility persistence index, the graphical user interface module proceed to step 327 where an additional condition is tested.

At step 327 the graphical user interface module determines whether the contraction medication information indicates that contraction inducing medication was given to the obstetrics patient. Optionally, step 327 may also evaluate the level (or dosage) of contraction inducing medication and use that information in effecting the decision step 327.

If step 327 is answered in the affirmative and the contraction medication information indicates that contraction inducing medication was given to the obstetrics patient, step 308 determines that an alarm event should be caused and the graphical user interface proceeds to step 310.

If step 327 is answered in the negative and the contraction medication information indicates that contraction inducing medication was not given to the obstetrics patient, the graphical user interface module proceed to step 329 where an additional condition is tested.

At step 329 the graphical user interface module determines whether the fetal heart rate information available indicates a problematic risk level associated with the baby's well-being. The fetal heart rate information may include a fetal heart rate signal or, alternatively, may include information conveying a level of risk associated with the fetus, the level of risk being derived on the basis of a fetal heart rate signal. In a specific example of implementation, the fetal heart rate information includes a fetal heart rate signal and is received from the fetal heart rate sensor 110 (shown in FIG. 1). Where the fetal heart rate information includes a fetal heart rate signal, step 329 includes processing the signal to determine a level of risk associated with the fetal heart rate signal. Any suitable method for assessing a level of risk on the basis of a fetal heart rate signal may be used. For example, the level of risk may be based on the frequency of the fetal heart rate, whether it is too high or too low for a certain period of time. Alternatively, the level of risk may be based on other suitable known methods. A non-limiting example of a method for providing an indication of the level of risk is described in U.S. Pat. No. 7,113,819, entitled “Method and apparatus for monitoring the condition of a fetus”, issued on Sep. 26, 2006 to E. Hamilton et al. and assigned to LMS Medical Systems Ltd. The contents of this document are incorporated herein by reference. Other suitable methods for assessing a level of risk on the basis of a fetal heart rate signal may be used without detracting from the spirit of the invention. Advantageously, step 329 allows the graphical user interface module to take into account the behaviour of the fetal heart rate, and therefore the response of the baby, in combination with the contraction rate when causing an alarm event.

If step 329 is answered in the affirmative and the fetal heart rate information indicates a problematic risk level associated with the baby's well-being, step 308 determines that an alarm event should be caused and the graphical user interface proceeds to step 310.

If step 329 is answered in the negative and the fetal heart rate information does no indicates a problematic risk level associated with the baby's well-being, step 308 determines that no alarm should be caused and the graphical user interface proceeds to step 300.

Although step 327 is shown in FIG. 6B as being performed prior to step 329, it is to be appreciated that in certain implementations the order these conditions are tested may be inverted without detracting from the spirit of the invention.

In the specific example of implementation shown in FIG. 6B, steps 326 327 and 329 are optional steps which may be included or omitted from specific implementations of the present invention. In addition, it will be appreciated in light of the present specification that other suitable manners of determining whether an alarm event should be caused on the basis of the contractility persistence index may be used without detracting from the spirit of the invention. As such, it should be understood that the example depicted in FIG. 6B was presented for the purpose of illustration only.

Returning now to FIG. 6A, if step 308 determines that an alarm event should be caused, the graphical user interface module proceeds to step 310 where an alarm event is triggered. Examples of alarm events were described previously in the specification. The graphical user interface module then returns to step 300 where the next segment of the contraction signal is received and subsequently processed.

If step 308 determines that no alarm event should be caused, the graphical user interface module returns to step 300 where the next segment of the contraction signal is received and subsequently processed.

As can be observed, the process illustrated in FIG. 6A is an iterative process whereby the steps are repeated as time progresses and as new segments of the contraction signal are received by the system. Over time, the system processes the contraction signal to derive a set of persistent contractility indices, where each contractility persistence index in the set is associated to a segment of the contraction signal.

Although the exemplary embodiment of the process implemented by the graphical user interface described with reference to FIGS. 6A and 6B made reference to a single alarm event presented in box 310, it will be appreciated that different types of alarm events may be caused by the graphical user interface. More specifically, different circumstances conveyed by the computed contractility persistence index, contraction medication information, fetal heart rate information and optionally other conditions may be associated to respective types of alarm events. Therefore, although the specification described causing a given alarm event, it should be understood that different types of alarm events may be caused and that the type of alarm event caused may be conditioned at least in part on the basis of the circumstances conveyed by the contractility persistence index, (optionally) contraction medication information, (optionally) fetal heart rate information and optionally other conditions.

### Variant

As a variant, the graphical user interface module is adapted for displaying, concurrently with the contractility persistence index additional information elements related to maternal and fetal well-being and/or labour progression.

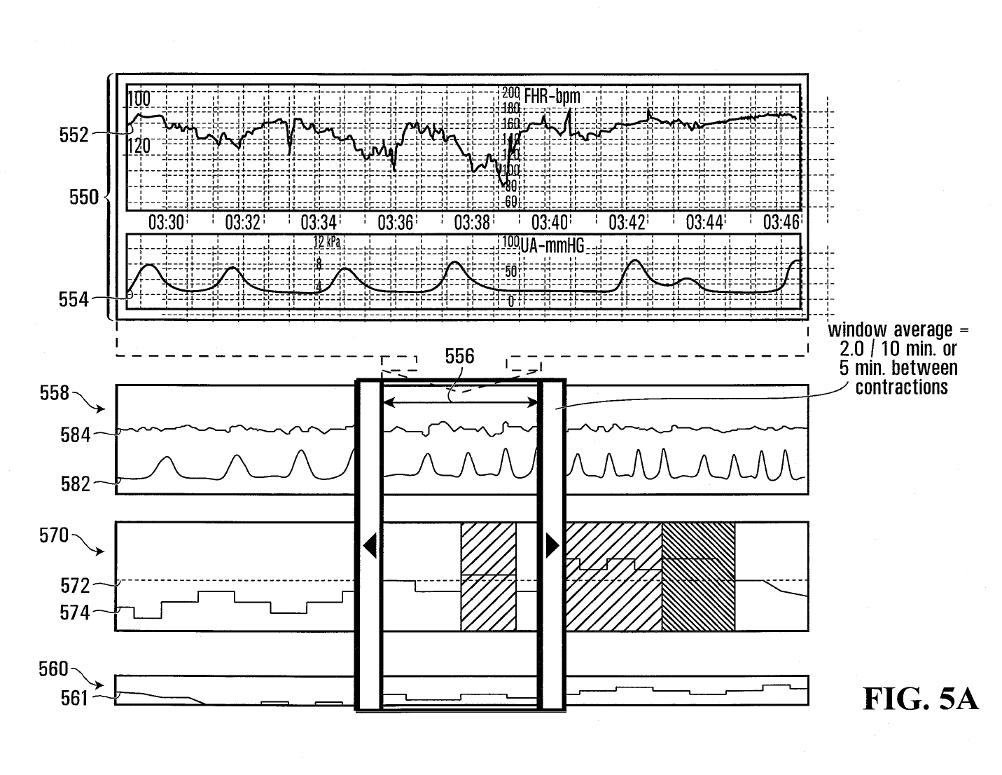

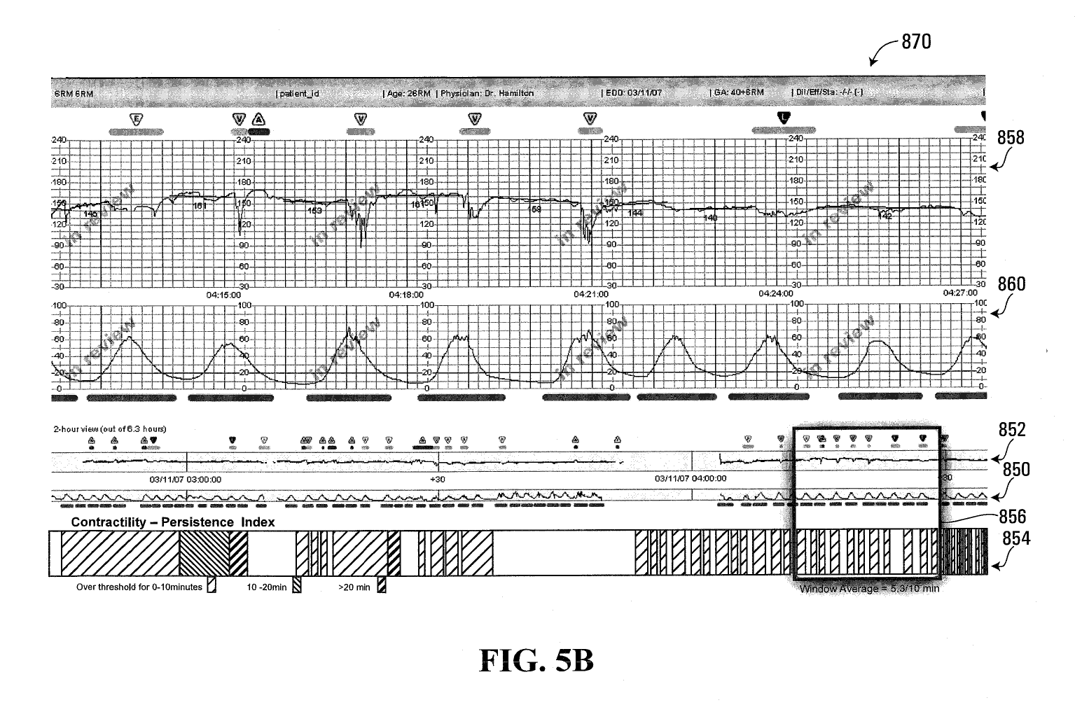

FIG. 5A of the drawings depicts a non-limiting example of implementation of a display generated by the graphical user interface module in accordance with this variant.

As shown, the graphical user interface module displays a first viewing window 570 conveying the value of the contractility persistence index over time. In this example a colour scheme is used to convey different values of the index over time. In this specific example, a first tracing 574 conveying rates of uterine contractions over time and a second tracing 572 conveying a threshold rate of uterine contractions are superposed onto the contractility persistence index in first viewing window 570. The graphical user interface module also displays a second viewing window 558 including a tracing 582 conveying a uterine contraction pattern over time (TOCO tracing) and a tracing 584 conveying a fetal heart rate pattern over time. The tracing 582 conveying a uterine contraction pattern over time is derived on the basis of the contraction signal received from the uterine activity sensor 120 (shown in FIG. 1). The tracing 584 conveying a fetal heart rate pattern over time is derived on the basis of the fetal heart rate signal received from the fetal heart rate sensor 110 (also shown in FIG. 1). Preferably, the first viewing window 570 and second viewing window 558 are time-aligned with one another on the display. In addition, it will be appreciated that either one of the tracings 584 and 582 may be omitted from the second viewing window 558 or that these tracings 584 and 582 may be displayed in separate viewing windows without detracting from the spirit of the invention. It will also be appreciated that the first tracing 574 conveying rates of uterine contractions over time and the second tracing 572 conveying a threshold rate of uterine contractions may be omitted from the first viewing window 570 in certain implementations.

Advantageously, the display of the tracing 584 conveying a fetal heart rate pattern over time allows the users of the system to view a representation of the baby's response to the contraction events.

The display of the tracing 582 conveying a uterine contraction pattern over time allows the users of the system to view a representation of the original contraction signal and to assess whether the tracing 574 conveying rates of uterine contractions over time accurately reflects the rate of contractions in the original contraction signal. This is particularly useful when the contraction signal generated by the uterine activity sensor part of the electronic fetal monitor 110 (shown in FIG. 1) is a continuous contraction signal (as opposed to a unitary contraction signal) since pattern recognition techniques must be used on such a continuous signal to detect the occurrence of a contraction event. Such pattern recognition techniques occasionally erroneously detect an occurrence of a contraction event or may fail to detect an occurrence of a contraction event. Therefore, by presenting the user with the tracing 582 conveying a uterine contraction pattern over time, that user may adjust his/her assessment of the first tracing 574.

In the embodiment depicted, the graphical user interface module also displays a control 556 allowing a user to select a portion of the tracings in the first viewing window 570 and/or the second viewing window 558. The user is enabled to manipulate the control 556 by providing signals using user input device 118 (shown in FIG. 1).

In a specific implementation, the control 556 includes a selection box having a transparent portion superposed upon the first viewing window 570 and the second viewing window 558. The portions of the tracings viewable through the transparent portion correspond to the selected portions. The control 556 allows the user to displace and modify the size of the selection box to select a portion of the tracings. Other manners in which portions of a signal may be selected are described in U.S. Pat. No. 6,907,284 issued to E. Hamilton et al. on Jun. 14, 2005. The contents of this document are incorporated herein by reference.

In the embodiment depicted, the control 556 is a sliding window superposed on the viewing windows 570 558 and 560. This sliding window is moveable along the time axis (x-axis) such as to allow the user to select a desired time segment. Optionally, and as shown in the embodiment depicted, the graphical user interface module also displays information related to the portion of the tracings selected by the control 556. Such information may include characteristics such as:
the average contraction rate in the time segment within the sliding window;
the periodicity (time interval between successive contractions) of the contraction rate in the time segment within the sliding window;
the average fetal heart rate in the time segment within the sliding window;
baseline level of fetal hear rate (FHR);
the variability of FHR.

It will be appreciated that the above examples of information is not intended to be an exhaustive list and that other types of information may be provided without detracting from the spirit of the invention.

In the embodiment depicted in FIG. 5A, the graphical user interface module also displays a third viewing window 550 including a tracing 552 conveying a fetal heart rate pattern over time and a tracing 554 conveying a uterine contraction pattern over time (TOCO tracing). The tracing 552 in the third viewing window 550 conveying a fetal heart rate pattern over time corresponds to the selected portion of the tracing 584 in the second viewing window 558 and is a zoomed-in view of that selected portion 556 The tracing 554 in the third viewing window 550 conveying a uterine contraction pattern over time (TOCO tracing) corresponds to the selected portion of the tracing 582 in the second viewing window 558 and is a zoomed-in view of that selected portion 556. In addition, it will be appreciated that either one of the tracings 552 and 554 may be omitted from the third viewing window 550 or that these tracings 552 and 554 may be displayed in separate viewing windows without detracting from the spirit of the invention.

Advantageously, by displaying zoomed-in views of the selected portions of the tracings 582 and 584, a user will be able to better view responses of the fetal heart rate to individual contraction events (amount of variability size and type of deceleration) and will be able to better assess the intensity and duration of a given contraction event.

In the embodiment depicted, the graphical user interface module also displays a fourth viewing window 560 including a tracing 561 conveying information associated to administration of contraction inducing medication to the obstetrics patient. The tracing 561 is derived on the basis of contraction medication information received by apparatus 100 (shown in FIG. 1). The contraction medication information may indicate whether contraction-inducing medication was administered and, optionally, a dosage of the contraction inducing medication administered. Since, typically, contraction inducing medication is administered continuously over time and not as a one shot dose, the contraction medication information when conveying a dosage of the contraction inducing medication administered may convey such dosage over time. In a first specific example of implementation, the contraction medication information is provided by the clinical staff using user-input device 118. In this first implementation, the clinical staff preferably enters the level (or dosage) of the contraction-inducing medication administered and updates that information when the dosage is modified. In a second specific example of implementation, the contraction medication information is provided automatically by a device, typically in the form of an electronic pump, designed to measure the dosage of medication provided to the obstetrics patient and provide that information over time to apparatus 100. In the embodiment depicted in FIG. 5A, the tracing 561 shows the level of medication administered over time to stimulate contractions. It will be appreciated by the person skilled in the art of obstetrics that the tracing 561 is not representative of an actual (real life) situation and that the levels of medication conveyed by tracing 561 are presented here for the purpose of illustration only.

Advantageously, the tracing 561 allows the clinical staff to readily view whether contraction inducing medication was administered to the obstetrics patient being monitored (and optionally the amount of contraction inducing medication that was administered).

FIG. 5B of the drawings depicts a screen capture of a specific practical example of implementation of a user interface for a labor monitoring system.

As shown, the graphical user interface module displays a viewing window 870 including a tracing 850 conveying a uterine contraction pattern over time (TOCO tracing) and a tracing 852 conveying a fetal heart rate pattern over time. The tracing 850 conveying a uterine contraction pattern over time is derived on the basis of the contraction signal received from the uterine activity sensor 120 (shown in FIG. 1). The tracing 852 conveying a fetal heart rate pattern over time is derived on the basis of the fetal heart rate signal received from the fetal heart rate sensor 110 (also shown in FIG. 1). The viewing window 870 also includes a graph 854 conveying the value of the contractility persistence index over time. In this example a colour scheme is to convey different values of the index over time.

The viewing window 870 also includes a control in the form of a selection box 856 having a transparent portion superposed on the tracings 850 852 and 854. The portions of the tracings or time signals viewable through the transparent portion correspond to selected portions of the tracings. The selection box 856 is moveable along the time axis (x-axis) such as to allow the user to select a desired time segment. The size of the selection box 856 can also be modified such as to include a larger portion of the tracings. Below the selection box 856 appears information related to characteristics of the tracings within the transparent portion. More specifically, in the example depicted, an indication of the average number of contraction within the viewing window is presented. When the window is displaced along the x-axis, the information appearing below is accordingly updated to reflect the characteristics of the new portion of the tracings selection by selection box 856.

The viewing window 870 also includes a tracing 858 conveying a fetal heart rate pattern over time and a tracing 860 conveying a uterine contraction pattern over time (TOCO tracing). The tracing 858 conveying a fetal heart rate pattern over time corresponds to the selected portion of the tracing 852 and is a zoomed-in view of the selected portion of tracing 852 selected by selection box 856. The tracing 860 conveying a uterine contraction pattern over time (TOCO tracing) corresponds to the selected portion of the tracing 850 in the second viewing window 558 and is a zoomed-in view of that selected portion.

### Specific Physical Implementation

Those skilled in the art should appreciate that in some embodiments of the invention, all or part of the functionality previously described herein with respect to the apparatus 100 (shown in FIG. 1) for implementing a user interface for displaying uterine contraction information may be implemented as pre-programmed hardware or firmware elements (e.g., application specific integrated circuits (ASICs), electrically erasable programmable read-only memories (EEPROMs), etc.), or other related components.

In other embodiments of the invention, all or part of the functionality previously described herein with respect to the apparatus 100 (shown in FIG. 1) for implementing a user interface for displaying uterine contraction information may be implemented as software consisting of a series of instructions for execution by a computing unit. The series of instructions could be stored on a medium which is fixed, tangible and readable directly by the computing unit, (e.g., removable diskette, CD-ROM, ROM, PROM, EPROM or fixed disk), or the instructions could be stored remotely but transmittable to the computing unit via a modem or other interface device (e.g., a communications adapter) connected to a network over a transmission medium. The transmission medium may be either a tangible medium (e.g., optical or analog communications lines) or a medium implemented using wireless techniques (e.g., microwave, infrared, RF or other transmission schemes).

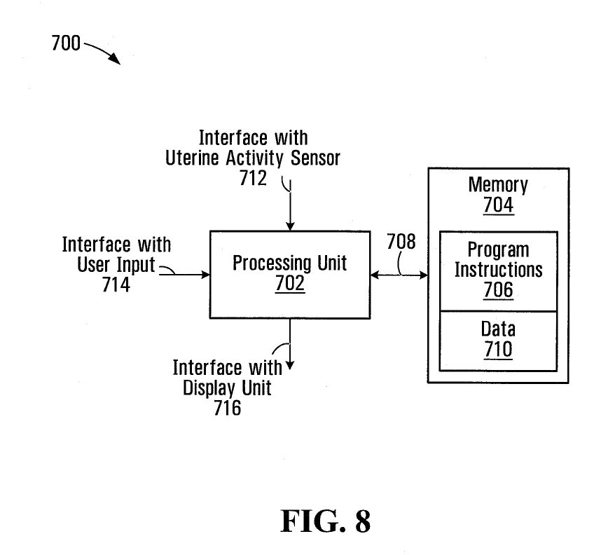

The apparatus 100 (shown in FIG. 1) implementing a user interface for displaying uterine contraction information may be configured as a computing unit of the type depicted in FIG. 8, including a processing unit 702 and a memory 704 connected by a communication bus 708. The memory 704 includes data 710 and program instructions 706. The processing unit 702 is adapted to process the data 710 and the program instructions 706 in order to implement the functional blocks described in the specification and depicted in the drawings. In a non-limiting implementation, the program instructions 706 implement the functionality of processing unit 206 described above with reference to FIG. 2. Amongst others, program instructions 706 may implement the process previously described with reference to FIGS. 6A and 6B. The computing unit 702 may also comprise a number of interfaces 712 714 716 for receiving or sending data elements to external devices. For example, interface 712 is used for receiving data streams indicative of uterine activity and interface 714 is used for receiving a control signals and/or information from the user. Interface 716 is for releasing a signal causing a display unit to display the user interface generated by the program instructions 706. Optionally, the computing unit 702 may include additional interfaces (not shown) for receiving information from additional sensors such as, for example, a fetal heart rate sensor. Alternatively, the computing unit 702 may include an interface (not shown) for receiving information originating from multiple sensors including but no limited to a fetal heart rate sensor and a uterine activity sensor. The computing unit shown in FIG. 8 may be part of any suitable computing device including, but not limited to, a desktop/laptop computing device or a portable digital assistant device (PDA).

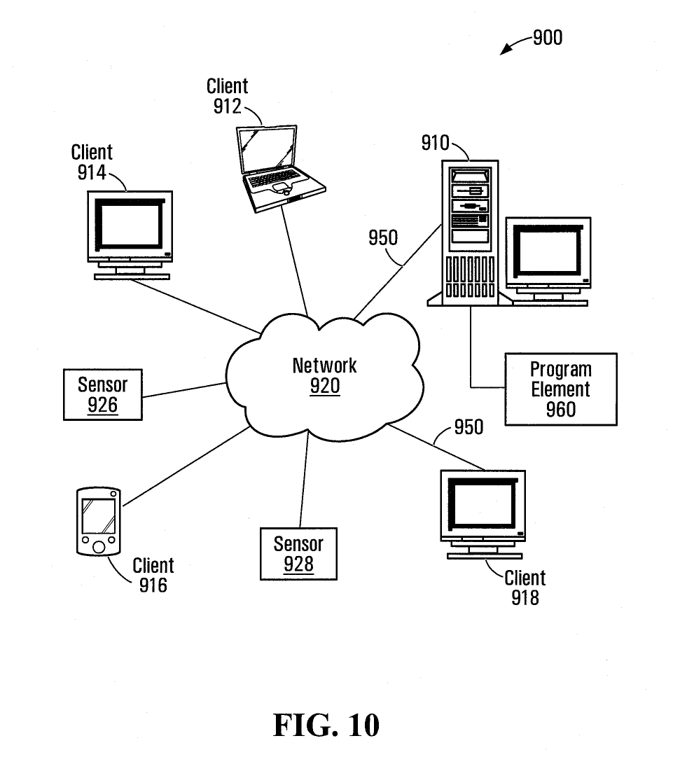

It will be appreciated that the system 150 depicted in FIG. 1 may also be of a distributed nature where the contraction signal is collected at one location by a uterine activity sensor and transmitted over a network to a server unit implementing the graphical user interface. The server unit may then transmit a signal for causing a display unit to display the graphical user interface. The display unit may be located in the same location as the uterine activity sensor, in the same location as the server unit or in yet another location. FIG. 10 illustrates a network-based client-server system 900 for displaying uterine contraction information. The client-server system 900 includes a plurality of client systems 912 914 916 918 connected to a server system 910 through network 920. The communication links 950 between the client systems 912 914 916 918 and the server system 910 can be metallic conductors, optical fibers or wireless, without departing from the spirit of the invention. The network 920 may be any suitable network including but not limited to a global public network such as the Internet and a private network. The server 910 may be adapted to process and issue signals to display multiple signals originating from multiple sensors 926 928 concurrently using suitable methods known in the computer related arts.

The server system 910 includes a program element 960 for execution by a CPU. Program element 960 implements similar functionality as program instructions 706 (shown in FIG. 8) and includes the necessary networking functionality to allow the server system 910 to communicate with the client systems 912 914 916 918 over network 920. In a non-limiting implementation, program element 960 includes a number of program element components, each program element components implementing a respective portion of the functionality of the user interface for displaying uterine contraction information.

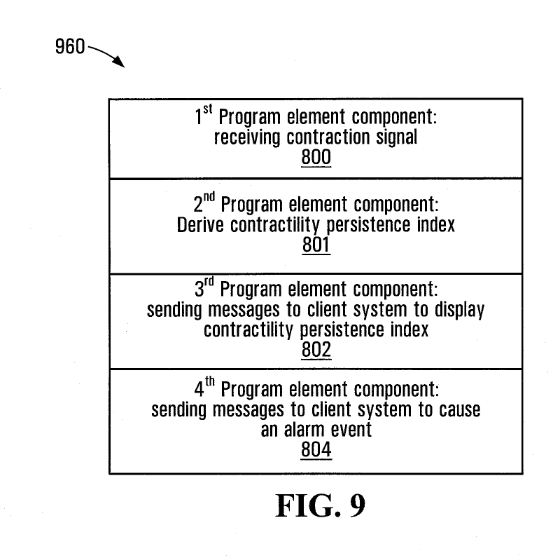

FIG. 9 shows a non-limiting example of the architecture of program element 960 at the server system. As shown, the program element 960 includes four program element components:
1. the first program element component 800 is executed on server system 910 and is for receiving a contraction signal conveying information related to occurrences of uterine contractions over time;
2. the second program element component 801 is executed on server system 910 and is for processing the contraction signal to derive a sequence of contractility persistence indices;
3. the third program element component 802 is executed on server system 910 and is for sending messages to a client system, say client system 914, for causing client system 914 to display the sequence of contractility persistence indices;
4. the fourth program element component 804, which is an optional component, is executed on server system 910 and is for selectively sending messages to client system 914 for causing an alarm event based on at least one contractility persistence index in the sequence of contractility persistence indices. Alternatively, the fourth program element component 804 is executed on server system 910 and is for selectively sending messages to a client system distinct from the client system 914 for causing an alarm event at the distinct client system. The messages for causing an alarm event may include alarm program elements for execution at the client system, the alarm program elements implementing the alarm events when executed at the client system. Alternatively, the alarm program elements for implementing the alarm events are stored at the client system and the messages for causing an alarm event transmitted from the server system 910 include instructions for causing the alarm program elements at the client system to be executed.

Those skilled in the art should further appreciate that the program instructions 706 and 960 may be written in a number of programming languages for use with many computer architectures or operating systems. For example, some embodiments may be implemented in a procedural programming language (e.g., “C”) or an object oriented programming language (e.g., “C++” or “JAVA”).

Although the present invention has been described in considerable detail with reference to certain preferred embodiments thereof, variations and refinements are possible without departing from the spirit of the invention. Therefore, the scope of the invention should be limited only by the appended claims and their equivalents.

1) A method for displaying uterine contraction information, said method comprising:
a) receiving at an input of a computing apparatus a contraction signal conveying information related to occurrences of uterine contractions over time;
b) using a processor at the computing apparatus, processing said contraction signal to derive a sequence of contraction persistence indices, each contraction persistence index in the sequence being associated to a portion of the contraction signal and conveying:
i) whether there is an excess in the rate of contraction in the associated portion of the contraction signal; and
ii) if there is an excess in the rate of contraction in the associated portion of the contraction signal, whether this excess is part of a sustained pattern of excess in the rate of contractions;
c) causing the sequence of contraction persistence indices to be conveyed to a user on a display device.

2) A method as defined in claim 1, wherein each contraction persistence index in the sequence of contraction indices being derived at least in part based on:
i) a first contraction rate, the first contraction rate being associated to a first portion of the contraction signal; and
ii) a second contraction rate, the second contraction rate being associated to a second portion of the contraction signal, at least part of the second portion of the contraction signal preceding in time the first portion of the contraction signal.

3) A method as defined in claim 2, said method comprising causing an alarm event based at least in part on at least one contraction persistence index in the sequence of contraction persistence indices.

4) A method as defined in claim 2, wherein the contraction signal is associated to an obstetrics patient, said method comprising:
a) receiving contraction medication information conveying information associated to administration of contraction inducing medication to the obstetrics patient;
b) causing an alarm event based at least in part:
i) on at least part of the sequence of contraction persistence indices; and; and
ii) on said contraction medication information.

5) A method as defined in claim 2, wherein the contraction signal is associated to an obstetrics patient, said method comprising:
a) receiving fetal heart rate information;
b) causing an alarm event based at least in part:
i) on at least part of the sequence of contraction persistence indices; and
ii) on said fetal heart rate information.

6) A method as defined in claim 2, wherein the contraction signal is associated to an obstetrics patient, said method comprising:
a) receiving fetal heart rate information;
b) receiving contraction medication information conveying information associated to administration of contraction inducing medication to the obstetrics patient;
c) causing an alarm event based at least in part:
i) on at least part of the sequence of contraction persistence indices;
ii) on said contraction medication information; and
iii) on said fetal heart rate information.

7) A method as described in claim 3, wherein said alarm event includes displaying a visual indicator.

8) A method as described in claim 3, wherein said alarm event includes causing an audio signal to be issued.

9) A method as described in claim 3, wherein said alarm event includes causing a message signal to be transmitted to a remote device.

10) A method as described in claim 9, wherein the remote device is a device selected from the set consisting of a PDA, telephone, pager and computing terminal.

11) A method as described in claim 9, wherein said alarm event includes causing a message signal to be transmitted over a network to the remote device.

12) A computer readable storage medium storing a program element suitable for execution by a CPU, said program element implementing a graphical user interface module for displaying uterine contraction information, said graphical user interface module being programmed for:
a) receiving a contraction signal conveying information related to uterine contractions over time;
b) processing said contraction signal to derive a sequence of contraction persistence indices, each contraction persistence index in the sequence being associated to a portion of the contraction signal and conveying:
i) whether there is an excess in the rate of contraction in the associated portion of the contraction signal; and
ii) if there is an excess in the rate of contraction in the associated portion of the contraction signal, whether this excess is part of a sustained pattern of excess in the rate of contractions;
c) causing the sequence of contraction persistence indices to be conveyed to a user on a display device.

13) An apparatus for implementing a user interface for displaying uterine contraction information, said apparatus comprising:
a) an input for receiving a contraction signal conveying information related to occurrences of uterine contractions over time;
b) a processing unit in communication with said input, said processing unit being operative for:
i) processing said contraction signal to derive a sequence of contraction persistence indices, each contraction persistence index in the sequence being associated to a portion of the contraction signal and conveying:
(1) whether there is an excess in the rate of contraction in the associated portion of the contraction signal; and
(2) if there is an excess in the rate of contraction in the associated portion of the contraction signal, whether this excess is part of a sustained pattern of excess in the rate of contractions;
ii) generating a signal for causing the sequence of contraction persistence indices to be conveyed to a user on a display device;
c) an output in communication with said processing unit, said output being suitable for releasing the signal generated by the processing unit.

14) (canceled)

15) A server system implementing a graphical user interface module for displaying uterine contraction information, said server system storing a program element for execution by a CPU, said program element comprising:
a) first program element component for receiving a contraction signal conveying information related to occurrences of uterine contractions over time;
b) second program element component for processing said contraction signal to derive a sequence of contraction persistence indices, each contraction persistence index in the sequence being associated to a portion of the contraction signal and conveying:
i) whether there is an excess in the rate of contraction in the associated portion of the contraction signal; and
ii) if there is an excess in the rate of contraction in the associated portion of the contraction signal, whether this excess is part of a sustained pattern of excess in the rate of contractions;
c) third program element component for generating and issuing a signal for displaying a graphical representation of the sequence of contraction persistence indices.

16) (canceled)

17) (canceled)

18) (canceled)

19) A computer readable storage medium as defined in claim 12, wherein each contraction persistence index in the sequence of contraction indices being derived at least in part based on:
i) a first contraction rate, the first contraction rate being associated to a first portion of the contraction signal; and
ii) a second contraction rate, the second contraction rate being associated to a second portion of the contraction signal, at least part of the second portion of the contraction signal preceding in time the first portion of the contraction signal.

20) A computer readable storage medium as defined in claim 19, said graphical user interface module being programmed for causing an alarm event based at least in part on at least one contraction persistence index in the sequence of contraction persistence indices.

21) A computer readable storage medium as defined in claim 19, wherein the contraction signal is associated to an obstetrics patient, said graphical user interface module being programmed for:
a) receiving contraction medication information conveying information associated to administration of contraction inducing medication to the obstetrics patient;
b) causing an alarm event based at least in part:
i) on at least part of the sequence of contraction persistence indices; and; and
ii) on said contraction medication information.

22) A computer readable storage medium as defined in claim 19, wherein the contraction signal is associated to an obstetrics patient, said graphical user interface module being programmed for:
a) receiving fetal heart rate information;
b) causing an alarm event based at least in part:
i) on at least part of the sequence of contraction persistence indices; and
ii) on said fetal heart rate information.

23) A computer readable storage medium as defined in claim 19, wherein the contraction signal is associated to an obstetrics patient, said graphical user interface module being programmed for:
a) receiving fetal heart rate information;
b) receiving contraction medication information conveying information associated to administration of contraction inducing medication to the obstetrics patient;
c) causing an alarm event based at least in part:
i) on at least part of the sequence of contraction persistence indices;
ii) on said contraction medication information; and
iii) on said fetal heart rate information.
